(19) United States
(12) Patent Application Publication
(10) Pub. No.: US 2024/0041310 A1
(43) Pub. Date: Feb. 8, 2024

(54) SYSTEM FOR DIAGNOSIS OF AN OTITIS MEDIA VIA A PORTABLE DEVICE

(71) Applicant: MASSACHUSETTS EYE AND EAR INFIRMARY, Boston, MA (US)

(72) Inventors: Christopher J. Hartnick, Boston, MA (US); Michael S. Cohen, Boston, MA (US); Matthew G. Crowson, Boston, MA (US); Fouzi Benboujja, Boston, MA (US)

(21) Appl. No.: 18/258,838

(22) PCT Filed: Dec. 22, 2021
(86) PCT No.: PCT/US2021/064980

§ 371 Date: Jun. 22, 2023

Related U.S. Application Data
(60) Provisional application No. 63/130,368, filed on Dec. 23, 2020.
(60) Provisional application No. 63/271,027, filed on Oct. 22, 2021.

(51) Int. Cl. A61B 1/227; G16H 30/20; G16H 40/67; G16H 50/20; A61B 1/00

(52) U.S. Cl. CPC A61B 1/227; G16H 30/20; G16H 40/67; G16H 50/20; A61B 1/000096

(57) ABSTRACT

Systems and methods are provided for diagnosing otitis media. The method includes receiving an image captured from an image sensor and determining an instruction for a user to adjust a position of the image sensor to bring the image sensor into alignment with a tympanic membrane of a subject. The determined instruction is to the user via an output device. A clinical parameter representing otitis media is determined from an image of the tympanic membrane of the subject at a predictive model. The clinical parameter is provided to the user via the output device.

### CROSS-REFERENCE TO RELATED APPLICATION

This application claims the benefit of each of U.S. Provisional Patent Application No. 63/130,368 filed on Dec. 23, 2020 and entitled AUTOMATIC DIAGNOSIS OF MIDDLE EAR EFFUSION USING ARTIFICIAL INTELLIGENCE, and U.S. Provisional Patent Application No. 63/271,027 filed on Oct. 22, 2021 and entitled SYSTEM FOR DIAGNOSIS OF AN OTITIS MEDIA VIA A PORTABLE DEVICE, which is incorporated herein by reference in its entirety.

### TECHNICAL FIELD

This disclosure relates to diagnostic systems, and is specifically directed to a system for diagnosis of otitis media via a portable device.

### BACKGROUND

Otitis media and its acute and chronic variants are among the most common childhood infections with at least 80% of all children experiencing at least one episode before the age of 3 contributing to over 5 to 10 million clinic visits and millions of antibiotic prescriptions each year in the United States. Given the prevalence of otitis media, tympanostomy tube placement is the most common pediatric surgical procedure performed annually in the United States, accounting for 667,000 procedures in 2006 alone. While advances in antibiotic strategies and the widespread adoption of the pneumococcal conjugate vaccine have lessened morbidity and mortality from otitis media, the disease process still confers a massive burden on global public health. Consequences of undiagnosed and untreated otitis media still include hearing loss, delayed language development, and morbidity from extracranial and intracranial complications. From a health care systems perspective, the cost of care for otitis media ranges from 3 to 5 billion dollars annually in the United States.

For decades, a technique for the consistent and accurate diagnosis of otitis media—both acute and chronic—has been elusive. This diagnostic challenge has produced an array of responses ranging from targeted educational programs for medical trainees, novel otoscopic approaches and techniques, using absorbance and acoustic admittance measurements, integration of audiometric adjuncts such as tympanometry and clinical trials comparing the effectiveness of one or more of these approaches. Despite these efforts, diagnostic accuracy has yet to consistently surpass 70% for all providers across the full spectrum of primary care, pediatrics, and otolaryngology. There are significant implications for such misdiagnoses. Undertreatment can lead to lead to possible morbid consequences such as mastoiditis, meningitis, and sensorineural hearing loss. Overtreatment may lead to excessive antibiotic usage and increased development of resistance, unnecessary tympanostomy tube procedures, and excess days off from school for children and work for parents, as well as significant deficits in quality of life for both children and parents. The persistent gap in diagnostic accuracy underscores a pressing need for innovation using an alternative approach.

### SUMMARY

In one example, a system includes at least one processor, an image sensor, an output device, and at least one non-transitory computer readable medium, storing executable instructions executable by the at least one processor. The executed instructions provide a guidance component configured to receive an image captured from the image sensor and determine an instruction for a user to adjust a position of the image sensor to bring the image sensor into alignment with a tympanic membrane of a subject. A user interface provides the determined instruction to the user via the output device, and a predictive model that determines a clinical parameter representing otitis media from an image of the tympanic membrane of the subject. The clinical parameter is provided to the user via the output device.

In another example, a method is provided for diagnosing otitis media. The method includes receiving an image captured from an image sensor and determining an instruction for a user to adjust a position of the image sensor to bring the image sensor into alignment with a tympanic membrane of a subject. The determined instruction is to the user via an output device. A clinical parameter representing otitis media is determined from an image of the tympanic membrane of the subject at a predictive model. The clinical parameter is provided to the user via the output device.

In a further example, another method is provided for diagnosing otitis media. A set of steps are iteratively repeated until a contour associated with the tympanic membrane is completely within a field of view of an image sensor. The iterative steps include receiving an image captured from the image sensor, identifying the contour associated with the tympanic membrane, determining an appropriate movement of the image sensor to being the contour associated with the tympanic membrane into a center of a field of view of the image sensor, and providing the determined instruction to the user via an output device. The image captured from the image sensor is then provided to a predictive model that determines a clinical parameter representing otitis media from the image captured at the image sensor. The clinical parameter is provided to the user via an output device.

### BRIEF DESCRIPTION OF THE DRAWINGS

FIG. 1 illustrates a system for diagnosing otitis media in an ear of a subject;

FIG. 2 depicts an example of a system for diagnosis of otitis media via a mobile device;

FIG. 3 illustrates one example of an optical assembly that can be used with the system of FIG. 2;

FIG. 4 illustrates one example of a method for diagnosis of otitis media;

FIG. 5 illustrates another example of a method for diagnosis of otitis media; and

FIG. 6 is a schematic block diagram illustrating an exemplary system of hardware components capable of implementing examples of the systems and methods disclosed in FIGS. 1-5.

### DETAILED DESCRIPTION

As used in this application, “patient data” refers to medically relevant data associated with the patient. This can include, but is not limited to, patient characteristics, such as age, sex, height, and weight; laboratory values such as blood glucose level, blood chemistry panels, complete blood counts, blood gas levels, urinalysis, culture results; and clinical measurements such as blood pressure, heart rate, oxygen saturation, exhalatory anesthetic agent volume and content, and respiratory rate, and medical history, including past values for clinical measurements and patient characteristics, diagnoses of medical conditions, allergies, and current and past therapeutic interventions performed on the patient.

A “therapeutic,” is a medication, an other therapeutic substance, such as blood or intravenous fluid, or a therapeutic action, which can include, but is not limited to, taking bloodwork, monitoring the patient, adjusting the sensors monitoring the patient, consulting other professional staff, suctioning the patient, adjusting pressure points, or starting chest compressions in cardiac arrest.

A “range” can have two bounding values (e.g., between five and ten milligrams) or a single explicit bounding value (e.g., less than ten milligrams).

An “alert” is a visual, auditory, or tactile notification provided to a user via an output device. An alert can, but does not necessarily, include information explaining a rationale for the alert or a prompt to allow the user to retrieve information related to the rationale for the alert.

A “predictive model” is a mathematical model or machine learning model that either predicts a future state of a parameter or estimates a current state of a parameter that cannot be directly measured.

“Light” is electromagnetic radiation in any of the visible, infrared, and ultraviolet spectra.

An “optical component” is a device formed from a material substantially transparent to light of a given wavelength that alter the state of light passing through or reflecting from the device.

This disclosure relates to systems and methods for diagnosing otitis media with a portable device. The device can include an output device, such as a display, that can be used by a guidance component to guide a user in positioning a portion of an imaging device. Once the tympanic membrane is determined to be present in a field of view of the imaging device, an image can be captured and provided to a predictive model for analysis. The predictive model provides a clinical parameter representing either the presence or absence of otitis media or the presence or absence of effusion or infection. In one example, the system can be implemented as software on a mobile device in combination with an optical assembly that is removably attached to the mobile device to facilitate acquisition of the image.

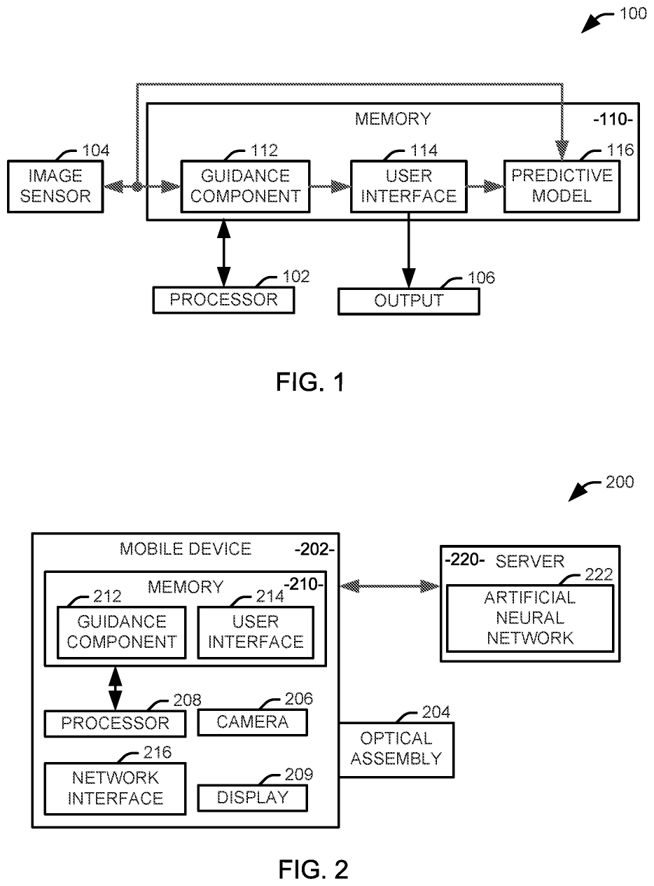

FIG. 1 illustrates a system 100 for diagnosing otitis media in an ear of a subject. The system 100 includes at least one processor 102, an image sensor 104, an output device 106, and at least one non-transitory computer readable medium 110 that stores executable instructions executable by a processor. The instructions include a guidance component 112 that is configured to receive an image captured from the image sensor and determine an instruction for a user to adjust a position of the image sensor to bring the image sensor into alignment with a tympanic membrane of a subject. A user interface 114 provides the determined instruction to the user via the output device. For example, the user can be instructed to tilt an extension of the system 100, for example, a speculum that is inserted into the ear, in a specific direction.

Once an image of the complete tympanic membrane is captured, it can be provided to a predictive model 116 that determines a clinical parameter representing otitis media from the image of the tympanic membrane of the subject. The predictive model 116 can be trained on training data representing the various classes or dependent variables of interest. In one example, the set of training data includes images taken from subjects who later underwent a myringotomy, with a class label for each image being determined from the myringotomy. The training process of the predictive model 116 will vary with its implementation, but training generally involves a statistical aggregation of training data into a set of parameters for the model. Any of a variety of techniques can be utilized for the models, including support vector machines, regression models, self-organized maps, k-nearest neighbor classification or regression, fuzzy logic systems, data fusion processes, boosting and bagging methods, rule-based systems, or artificial neural networks. Many of these techniques require an explicit extraction of numerical features from the image, although certain techniques, such as convolutional neural networks and other deep learning approaches, can act directly on a set of chromatic values associated with each pixel of the image, with the feature extraction performed by convolutional layers within the neural network.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. An SVM classifier utilizes a user-specified kernel function to organize training data within a defined feature space. In the most basic implementation, the kernel function can be a radial basis function, although the systems and methods described herein can utilize any of a number of linear or non-linear kernel functions.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier.

Classic ANN classifiers are fully connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A k-nearest neighbor model populates a feature space with labelled training samples, represented as feature vectors in the feature space. In a classifier model, the training samples are labelled with their associated class, and in a regression model, the training samples are labelled with a value for the dependent variable in the regression. When a new feature vector is provided, a distance metric between the new feature vector and at least a subset of the feature vectors representing the labelled training samples is generated. The labelled training samples are then ranked according to the distance of their feature vectors from the new feature vector, and a number, k, of training samples having the smallest distance from the new feature vector are selected as the nearest neighbors to the new feature vector.

In one example of a classifier model, the class represented by the most labelled training samples in the k nearest neighbors is selected as the class for the new feature vector. In another example, each of the nearest neighbors can be represented by a weight assigned according to their distance from the new feature vector, with the class having the largest aggregate weight assigned to the new feature vector. In a regression model, the dependent variable for the new feature vector can be assigned as the average (e.g., arithmetic mean) of the dependent variables for the k nearest neighbors. As with the classification, this average can be a weighted average using weights assigned according to the distance of the nearest neighbors from the new feature vector. It will be appreciated that k is a metaparameter of the model that is selected according to the specific implementation. The distance metric used to select the nearest neighbors can include a Euclidean distance, a Manhattan distance, or a Mahalanobis distance.

A regression model applies a set of weights to various functions of the extracted features, most commonly linear functions, to provide a continuous result. In general, regression features can be categorical, represented, for example, as zero or one, or continuous. In a logistic regression, the output of the model represents the log odds that the source of the extracted features is a member of a given class. In a binary classification task, these log odds can be used directly as a confidence value for class membership or converted via the logistic function to a probability of class membership given the extracted features.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used, but a continuous parameter can be computed according to a number of decision trees that select a given task. It will be appreciated that the number of trees, as well as a number of features used to generate trees, can be selected as metaparameters for the random forest model.

In some implementations, some or all of the predictive model 116 can be implemented using a quantum computer. For example, many of the models described are, in practice, vectorized for more efficient computation and require manipulation of large matrices and vectors. The inputs and outputs of these computations can be represented as quantum amplitudes and a quantum algorithm can be performed to simulate the result of the computation. Other quantum algorithms, such as quantum annealing, can be used for optimization tasks, which are common in training predictive models such as those described above. Finally, a number of quantum predictive models, such as quantum neural networks, hidden quantum Markov models, and quantum-enhanced reinforcement learning can be applied as part or all of the predictive model.

The predictive model 116 provides the clinical parameter representing otitis media to the user at the output device 106 via the user interface 114. It will be appreciated that the clinical parameter can be categorical or continuous. For example, the clinical parameter can be any of a continuous parameter representing a likelihood of otitis media within the subject, a categorical parameter having a first value representing the presence of otitis media and a second value representing the absence of otitis media, a categorical parameter having a first value representing the absence of effusion, a second value representing partial effusion, and a third value representing complete effusion, a continuous parameter representing a likelihood of infection, and a categorical parameter having a first value representing the absence of infection, a second value representing possible infection, and a third value representing the presence of infection. For many applications, a binary categorical classification of “normal” and “abnormal” allows for a straightforward screening of subjects' ears, allowing any subjects for whom the ear appears abnormal to be brought to a physician or other medical profession for further diagnosis.

FIG. 2 depicts an example of a system 200 for diagnosis of otitis media via a mobile device 202. The system 200 includes an optical assembly 204 that attaches to the mobile device and facilitates capture of images of the tympanic membrane of a patient. The optical assembly 204 can include a tapered end for insertion into the ear, a light source for illuminating the tympanic membrane, and appropriate optical components to correct spatial and chromatic aberrations before an image of the tympanic membrane is captured at a camera 206 of the mobile device 202.

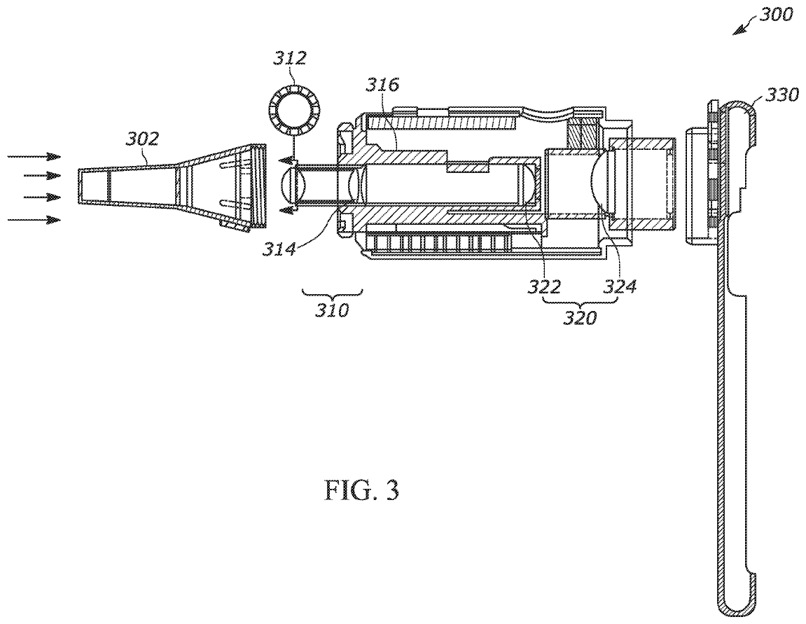

FIG. 3 illustrates one example of an optical assembly 300 that can be used with the system of FIG. 2. The optical assembly 300 includes a speculum 302 with a conically tapering shape. In some implementations, the speculum 302 can be single use. In other implementations, the speculum 302 can contain optical components and be reused between patients after appropriate sterilization. The speculum 302 can be removably attached to a first optics module 310 that includes optical elements, such as lenses 312 and 314, to improve the quality of images and enhance the depth of field during image acquisition. In some implementations, at least one optical component (e.g., 312) from the first optics module 310 can extend into speculum 302 when the speculum is attached to the first optics module 310. In the illustrated implementation, the first optics module 310 includes two lenses 312 and 314, as well as a light source 216 for illuminating the interior of the auditory canal. In one implementation, the light source 316 is implemented as an LED light.

A second optical module 320 is removably attached to the first optical module 310. The second optical module 320 contains optical elements 322-324 for improving quality of captured images of the tympanic membrane. The second optical module 320 is removable attached to a case 330 designed to removably attach to the mobile device 202. It will be appreciated that the mobile device 202 can include optical components with the camera 206. In one example, the optical modules 310 and 320 can be translated relative to one another, the speculum 302, and the case 320. In one implementation, the optical modules 310 and 320 can be removed and replaced with other optical modules (not shown) having different optical components. For example, the other optical modules can include appropriate optical components for filtering different wavelengths for imaging, adjusting the assembly for different focal depths, or providing different magnifications.

Returning to FIG. 2, the mobile device 202 includes a processor 208, a display 209, and a storage medium 210 that stores a guidance component 212 that guides a user through capture of an image of the tympanic membrane. Specifically, the guidance component 212 works in tandem with a user interface 214 of the mobile device 202 to provide instructions to a user to improve the quality of an image captured using the mobile device 202 and optical assembly 204. It will be appreciated that most mobile devices, during the image capture process, continuously acquire and display video until a user instructs the device to capture an image or segment of video. The guidance component 212 can receive sample frames from this video and evaluate the image to determine whether the tympanic membrane is within the frame. Alternatively, the system can wait for an image capture initiated by the user before capturing the image.

In the illustrated implementation, the guidance component 212 applies an edge detection algorithm to identify a contour associated with the tympanic membrane. Once the contour has been identified, the shape of the contour can be evaluated to determine an appropriate movement of the camera to bring the contour associated with the tympanic membrane into a center of a field of view of the camera 206. An image can then be captured with the tympanic membrane fully within the field of view of the camera 206 and provide to a remote server 220 via a network interface 216 associated with the mobile device. It will be appreciated that the server 220 can include appropriate hardware for implementing a server including a processor, memory, and a network interface for connecting the device to a local or wide area network.

In the illustrated implementation, the captured image can be evaluated at an artificial neural network 222 hosted at the server 220. The artificial neural network 222 can be trained on training images captured from patients whose diagnosis is confirmed via a myringotomy procedure, with each image labeled as either “normal” or “abnormal”. In one example, the artificial neural network 222 is implemented as a convolutional neural network. In this implementation, a plurality of layers of the convolutional neural network can have weights established via transfer learning, and one or more weights near the output of the network are trained using the training images. The clinical parameter provided by the artificial neural network 222 can be sent back to the mobile device 202 for display to the user at the display 209.

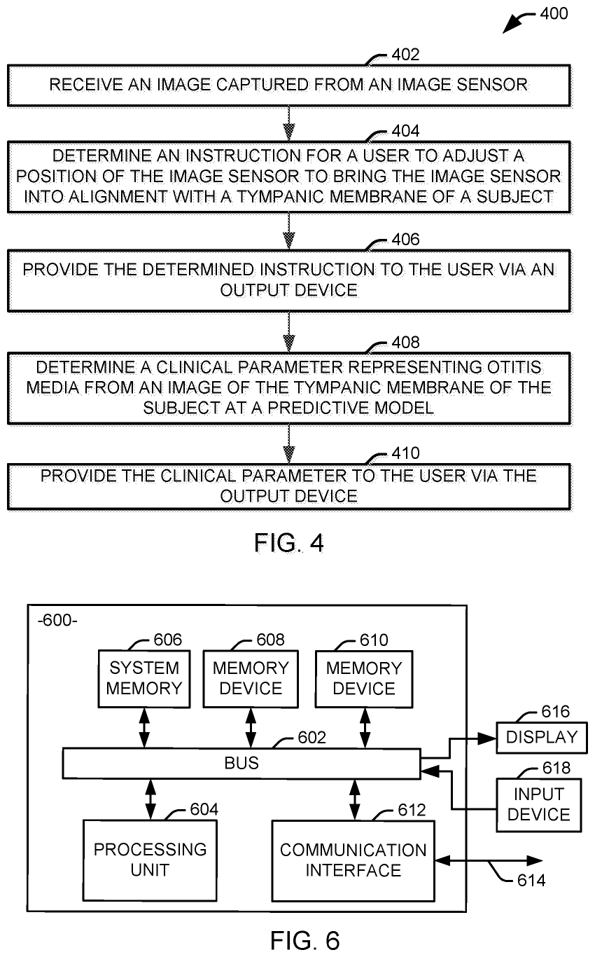

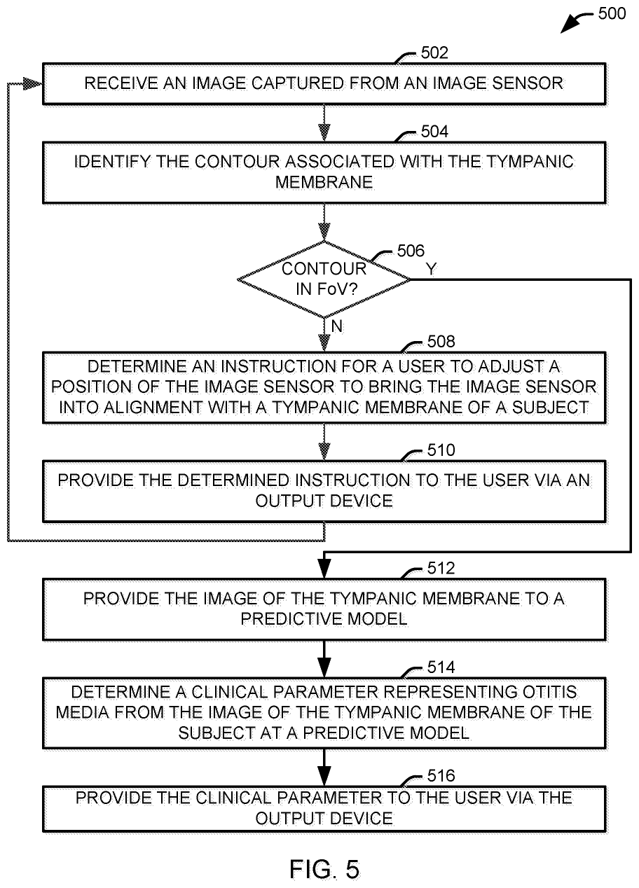

In view of the foregoing structural and functional features described above in FIGS. 1-3, example methods will be better appreciated with reference to FIGS. 4 and 5. While, for purposes of simplicity of explanation, the methods of FIGS. 4 and 5 are shown and described as executing serially, it is to be understood and appreciated that the present invention is not limited by the illustrated order, as some actions could in other examples occur in different orders and/or concurrently from that shown and described herein.

FIG. 4 illustrates one example of a method 400 for diagnosis of otitis media. At 402, an image is received from an image sensor. In one implementation, the image is captured at a camera associated with a mobile device using an optical assembly removably attached to the mobile device having a conical speculum for imaging within the ear. In this implementation, the image is obtained by attaching the optical assembly to the mobile device and inserting a distal end of the optical assembly into an ear of the patient. In another implementation, the image sensor and the optical assembly are integral as part of a standalone imaging system. At 404, an instruction is determined for a user to adjust a position of the image sensor to bring the image sensor into alignment with a tympanic membrane of a subject. In one implementation, a contour of the tympanic membrane is located via an edge detection algorithm, and the shape of the contour is used to direct the user to tilt the assembly containing the sensor until the contour is completely within the field of view of the image sensor.

At 406, the determined instruction is provided to the user via the output device. This can be accomplished via an output device, such as a display, speaker, or tactile feedback element associated with the imaging system, or a display of the mobile device. At 408, a clinical parameter representing otitis media is determined from an image of the tympanic membrane of the subject at a predictive model. In one implementation, the predictive model is a convolution neural network trained on images verified during a surgical procedure. The clinical parameter can include, for example, any of a continuous parameter representing a likelihood of otitis media within the subject, a categorical parameter having a first value representing the presence of otitis media and a second value representing the absence of otitis media, a categorical parameter having a first value representing the absence of effusion, a second value representing partial effusion, and a third value representing complete effusion, a continuous parameter representing a likelihood of infection, and a categorical parameter having a first value representing the absence of infection, a second value representing possible infection, and a third value representing the presence of infection. At 410, the clinical parameter is provided to the user via the output device.

FIG. 5 illustrates another example of a method 500 for diagnosis of otitis media. At 502, an image is received from an image sensor. At 504, a contour associated with the tympanic membrane is identified. In one implementation, an edge detection algorithm is applied to identify the contour of the tympanic membrane. At 506, it is determined if the contour associated with the tympanic membrane is completely within a field of view (FoV) of the image sensor. If not (N), an instruction is determined for a user to adjust a position of the image sensor to bring the image sensor into alignment with a tympanic membrane of a subject at 508. In one example, a shape of the contour is compared to an expected shape of the contour locate a missing portion of the contour, with the adjustment made to translate the field of view in the direction of the missing portion of the contour. At 510, the determined instruction is provided to the user via the output device, and the method returns to 502 to acquire another image from the image sensor.

If the contour associated with the tympanic membrane is completely within a field of view (FoV) of the image sensor (Y), the method advances to 512, where the image captured from the image sensor is provided to a predictive model. In one implementation, the image captured from the image sensor is send to a remote server that hosts the predictive model via a network interface. At 514, the predictive model determines a clinical parameter representing otitis media from the image captured at the image sensor. The clinical parameter is provided to the user via an output device at 516.

FIG. 6 is a schematic block diagram illustrating an exemplary system 600 of hardware components capable of implementing examples of the systems and methods disclosed in FIGS. 1-5. The system 600 can include various systems and subsystems. The system 600 can be a personal computer, a laptop computer, a workstation, a computer system, an appliance, an application-specific integrated circuit (ASIC), a server, a server blade center, a server farm, etc.

The system 600 can includes a system bus 602, a processing unit 604, a system memory 606, memory devices 608 and 610, a communication interface 612 (e.g., a network interface), a communication link 614, a display 616 (e.g., a video screen), and an input device 618 (e.g., a keyboard and/or a mouse). The system bus 602 can be in communication with the processing unit 604 and the system memory 606. The additional memory devices 608 and 610, such as a hard disk drive, server, stand-alone database, or other non-volatile memory, can also be in communication with the system bus 602. The system bus 602 interconnects the processing unit 604, the memory devices 606-610, the communication interface 612, the display 616, and the input device 618. In some examples, the system bus 602 also interconnects an additional port (not shown), such as a universal serial bus (USB) port.

The processing unit 604 can be a computing device and can include an application-specific integrated circuit (ASIC). The processing unit 604 executes a set of instructions to implement the operations of examples disclosed herein. The processing unit can include a processing core.

The additional memory devices 606, 608, and 610 can store data, programs, instructions, database queries in text or compiled form, and any other information that can be needed to operate a computer. The memories 606, 608 and 610 can be implemented as computer-readable media (integrated or removable) such as a memory card, disk drive, compact disk (CD), or server accessible over a network. In certain examples, the memories 606, 608 and 610 can comprise text, images, video, and/or audio, portions of which can be available in formats comprehensible to human beings. Additionally or alternatively, the system 600 can access an external data source or query source through the communication interface 612, which can communicate with the system bus 602 and the communication link 614.

In operation, the system 600 can be used to implement one or more parts of a clinical decision support system in accordance with the present invention. Computer executable logic for implementing the clinical decision support system resides on one or more of the system memory 606, and the memory devices 608, 610 in accordance with certain examples. The processing unit 604 executes one or more computer executable instructions originating from the system memory 606 and the memory devices 608 and 610. The term “computer readable medium” as used herein refers to any medium that participates in providing instructions to the processing unit 604 for execution, and it will be appreciated that a computer readable medium can include multiple computer readable media each operatively connected to the processing unit.

Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to portable or fixed storage devices, optical storage devices, wireless channels, and/or various other storage mediums capable of storing that contain or carry instruction(s) and/or data.

What have been described above are examples of the invention. It is, of course, not possible to describe every conceivable combination of components or methodologies, but one of ordinary skill in the art will recognize that many further combinations and permutations of the invention are possible. Accordingly, the invention is intended to embrace all such alterations, modifications, and variations that fall within the scope of the appended claims and the application. Additionally, where the disclosure or claims recite “a,” “an,” “a first,” or “another” element, or the equivalent thereof, it should be interpreted to include one or more than one such element, neither requiring nor excluding two or more such elements. As used herein, the term “includes” means includes but not limited to, the term “including” means including but not limited to. The term “based on” means based at least in part on.

What is claimed is:

1. A system comprising:
at least one processor;
an image sensor;
an output device; and
at least one non-transitory computer readable medium, storing executable instructions executable by the at least one processor to provide:
a guidance component configured to receive an image captured from the image sensor and determine an instruction for a user to adjust a position of the image sensor to bring the image sensor into alignment with a tympanic membrane of a subject;
a user interface that provides the determined instruction to the user via the output device; and
a predictive model that determines a clinical parameter representing otitis media from an image of the tympanic membrane of the subject;
wherein the clinical parameter is provided to the user via the output device.

2. The system of claim 1, further comprising an optical assembly, comprising a plurality of optical elements aligned with the image sensor to improve a quality of images acquired at the image sensor.

3. The system of claim 2, wherein the optical assembly is removably attached to the image sensor.

4. The system of claim 3, wherein each of the processor, the image sensor, the output device, and the non-transitory computer readable medium are part of a mobile device, the optical assembly being configured to attach to a surface of the mobile device.

5. The system of claim 2, wherein the plurality of optical elements comprise a first set of optical elements in a first module and a second set of optical elements in a second module, the first module being removably attached to the image sensor and the second module being configured to removably attach to the first module at a first location.

6. The system of claim 5, wherein the second module has an associated first optical property and the system further comprising a third module, has an associated second property and configured to removably attach to the first module at the first location, such that the second module can be replaced with the third module to change an optical property of the optical assembly.

7. The system of claim 1, wherein the predictive model is implemented as an artificial neural network.

8. The system of claim 7, wherein the artificial neural network has a set of associated parameters generated from a stored set of training data, the set of training data comprising images taken from subjects who later underwent a myringotomy, a class label for each image being determined from the myringotomy.

9. The system of claim 1, wherein a first non-transitory computer medium of the at least one non-transitory computer readable medium and a first processor of the at least one processor are local to the image sensor, and a second non-transitory computer medium of the at least one non-transitory computer readable medium and a second processor of the at least one processor provide a server in a location remote from the image sensor, the system further comprising a first network interface associated with the first processor and a second network interface associated with the second processor, wherein the guidance component and the user interface are stored on the first non-transitory computer readable medium, the predictive model is stored on the second non-transitory computer readable medium, and the image of the tympanic membrane of the subject is provided to the predictive model via the first and second network interfaces.

10. The system of claim 1, wherein the guidance identifies a contour associated with the tympanic membrane via an edge detection algorithm and determines an appropriate movement of the image sensor to being the contour associated with the tympanic membrane into a center of a field of view of the image sensor.

11. A method comprising:
receiving an image captured from an image sensor;
determining an instruction for a user to adjust a position of the image sensor to bring the image sensor into alignment with a tympanic membrane of a subject;
providing the determined instruction to the user via an output device;
determining a clinical parameter representing otitis media from an image of the tympanic membrane of the subject at a predictive model; and
providing the clinical parameter to the user via the output device.

12. The method of claim 11, wherein the clinical parameter is one of a continuous parameter representing a likelihood of otitis media within the subject and a categorical parameter having a first value representing the presence of otitis media and a second value representing the absence of otitis media.

13. The method of claim 11, wherein the clinical parameter is a categorical parameter representing effusion, the categorical parameter having a first value representing the absence of effusion, a second value representing partial effusion, and a third value representing complete effusion.

14. The method of claim 11, further comprising:
attaching an optical assembly to a mobile device, the image sensor being contained within the mobile device; and
inserting a distal end of the optical assembly into an ear of the patient.

15. The method of claim 11, wherein determining the instruction for the user to adjust a position of the image sensor to bring the image sensor into alignment with a tympanic membrane of a subject comprises iteratively repeating the following steps until a contour associated with the tympanic membrane is completely within a field of view of an image sensor;
receiving an image captured from the image sensor;
identifying the contour associated with the tympanic membrane; and
determining an appropriate movement of the image sensor to being the contour associated with the tympanic membrane into a center of a field of view of the image sensor.

16. A method comprising:
iteratively repeating the following steps until a contour associated with the tympanic membrane is completely within a field of view of an image sensor;
receiving an image captured from the image sensor;
identifying the contour associated with the tympanic membrane;
determining an appropriate movement of the image sensor to being the contour associated with the tympanic membrane into a center of a field of view of the image sensor; and
providing the determined instruction to the user via an output device;
providing the image captured from the image sensor to a predictive model;
determining a clinical parameter representing otitis media from the image captured at the image sensor at the predictive model; and
providing the clinical parameter to the user via the output device.

17. The method of claim 16, determining the clinical parameter representing otitis media from the image captured at the image sensor comprises providing the image captured at the image sensor to a convolutional neural network trained on images taken from subjects who later underwent a myringotomy, a class label for each image being determined from the myringotomy.

18. The method of claim 16, wherein providing the image captured from the image sensor to a predictive model comprises sending the image captured from the image sensor to a remote server via a network interface.

19. The method of claim 16, further comprising:
attaching an optical assembly to a mobile device, the image sensor being contained within the mobile device; and
inserting a distal end of the optical assembly into an ear of the patient.

20. The method of claim 16, wherein the clinical parameter is one of a continuous parameter representing a likelihood of infection and a categorical parameter representing infection, the categorical parameter having a first value representing the absence of infection, a second value representing possible infection, and a third value representing the presence of infection.
